(19) United States
(12) Patent Application Publication
(10) Pub. No.: US 2022/0166170 A1
(43) Pub. Date: May 26, 2022

(54) Electrically Grounded Clothing and Blanket

(71) Applicant: No Choice, LLC, St. Petersburg, FL (US)

(72) Inventor: J. Mitchell Gordon, St. Petersburg, FL (US)

(73) Assignee: No Choice, LLC, St. Petersburg, FL (US)

(21) Appl. No.: 17/103,897

(22) Filed: Nov. 24, 2020

(51) Int. Cl. H01R 13/652; H01R 33/05; H01R 13/11; H01R 4/48; A41D 1/00

(52) U.S. Cl. CPC H01R 13/652; H01R 33/05; A41D 1/005; H01R 4/48; H01R 13/111

(57) ABSTRACT

An electrically grounded clothing item includes: a grounding wire configured to provide electrical grounding; electrically conductive thread configured to ground the entire garment through the grounding wire; and a conducting connector to which the grounding wire can be electrically connected at a first grounding wire end, the conducting connector configured to hold the grounding wire tightly to the electrically grounded clothing item, the grounding wire electrically connected at a second grounding wire end to an electrical ground.

### SUMMARY

An electrically grounded clothing item includes: a grounding wire configured to provide electrical grounding; electrically conductive thread configured to ground the entire garment through the grounding wire; and a conducting connector to which the grounding wire can be electrically connected at a first grounding wire end, the conducting connector configured to hold the grounding wire tightly to the electrically grounded clothing item, the grounding wire electrically connected at a second grounding wire end to an electrical ground.

An electrically grounded blanket includes: a grounding wire configured to provide electrical grounding; electrically conductive thread configured to ground the entire blanket through the grounding wire; and a conducting connector to which the grounding wire can be electrically connected at a first grounding wire end, the conducting connector configured to hold the grounding wire tightly to the electrically grounded clothing item, the grounding wire electrically connected at a second grounding wire end to an electrical ground.

### DESCRIPTION OF THE DRAWINGS

The accompanying drawings provide visual representations which will be used to more fully describe various representative embodiments and can be used by those skilled in the art to better understand the representative embodiments disclosed herein and their inherent advantages. In these drawings, like reference numerals identify corresponding elements.

FIGS. 1A-1B are a set of two drawings of an electrically grounded hoodie.

FIGS. 2A-2B are a set of two drawings of an electrically grounded pants.

FIGS. 3A-3B are a set of two drawings of an electrically grounded shirt.

FIGS. 4A-4B are a set of two drawings of an electrically grounded ski mask.

FIGS. 5A-5B are a set of two drawings of an electrically grounded cap.

FIGS. 6A-6B are a set of two drawings of an electrically grounded long underwear.

FIGS. 7A-7B are a set of two drawings of an electrically grounded blanket.

### DETAILED DESCRIPTION

Embodiments of the invention provide electrically grounded clothing. For example, the clothing comprises one or more of a hoodie, pants, a shirt, a ski mask, a cap, and long underwear.

Further embodiments of the invention provide an electrically grounded blanket.

The electrically grounded clothing comprises a grounding wire configured to provide electrical grounding. The electrically grounded clothing further comprises electrically conductive thread configured to electrically ground the entire garment through the grounding wire. For example, the grounding wire has a length between approximately five feet and approximately twenty feet.

The electrically grounded clothing further comprises a conducting connector to which the grounding wire can be electrically connected at a first grounding wire end. The conducting connector is configured to hold the grounding wire tightly. Preferably, but not necessarily, the conducting connector is sewn into the electrically grounded clothing. Alternatively, the conducting connector is physically attached to the electrically grounded clothing. In the case where the electrically grounded item comprises an electrically grounded blanket instead of electrically grounded clothing, the conducting connector is sewn into the electrically grounded clothing. Alternatively, the conducting connector is physically attached to the electrically grounded blanket.

For example, the conducting connector comprises a button sewn into the electrically grounded clothing. The button comprises a snap to which the grounding wire attaches. When the electrically grounded clothing is being worn but the grounding feature is not being used, a user conveniently can place the grounding wire in the user's pocket. Alternatively, or additionally, the conducting connector comprises a clamp. Alternatively, or additionally, the conducting connector comprises another device other than a clamp or button configured to hold the grounding wire tightly.

The grounding wire is electrically connected at a second grounding wire end to an electrical ground.

For example, the electrical ground comprises one or more of a ground pin on a three-prong outlet, a ground pin on an outlet other than a three-prong outlet, a direct connection to the ground, and a connection to a grounding device comprised in an electrical appliance.

For example, the three-prong outlet comprises one or more of a ground fault circuit interrupter (GFCI) outlet, a ground fault interrupter (GFI) outlet, an arc fault circuit interrupter (AFCI) outlet, and a smart outlet.

The electrically grounded clothing further comprises electrically conductive thread configured to ground the entire garment through the grounding wire. For example, the electrically conductive thread comprises one or more of silver, nickel, copper, gold, and another conductive material.

For example, the electrically conductive thread comprises high strength silver-coated nylon sewing thread manufactured by Suzhou Tek Silver Fiber Technology Co., Ltd. (http://mlsilver-fiber.com), of Suzhou, Jiangsu; China. For example, the electrically conductive thread can be used as a liner for electrically grounded pants.

Preferably, but not necessarily, the conducting connector is located near a user's waist line. Preferably, but not necessarily, for one or more of an electrically grounded shirt and an electrically grounded hoodie, the conducting connector is located near a bottom of the electrically grounded clothing item. Preferably, but not necessarily, for one or more of pants and long underwear pants, the conducting connector is located near a top of the clothing item. For example, the long underwear pants comprise long woolen underwear pants.

For example, the conducting connector is preferably located within six inches of the user's waist line. For example, the conducting connector is preferably located within twelve inches of the user's waist line.

Preferably, but not necessarily, for one or more of an electrically grounded shirt and an electrically grounded hoodie, the conducting connector is located within six inches of a bottom of the shirt. Alternatively, for one or more of the electrically grounded shirt and the electrically grounded hoodie, the conducting connector is located within twelve inches of a bottom of the electrically grounded clothing item.

Preferably, but not necessarily, for one or more of pants and long underwear pants, the conducting connector is located within six inches of a top of the electrically grounded clothing item. Alternatively, for one or more of pants and long underwear pants, the conducting connector is located within twelve inches of a top of the electrically grounded clothing item.

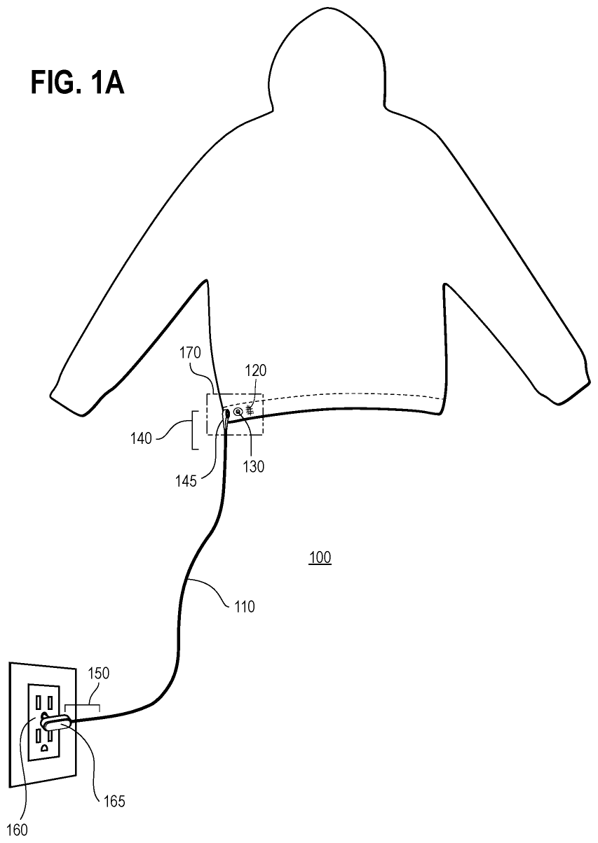

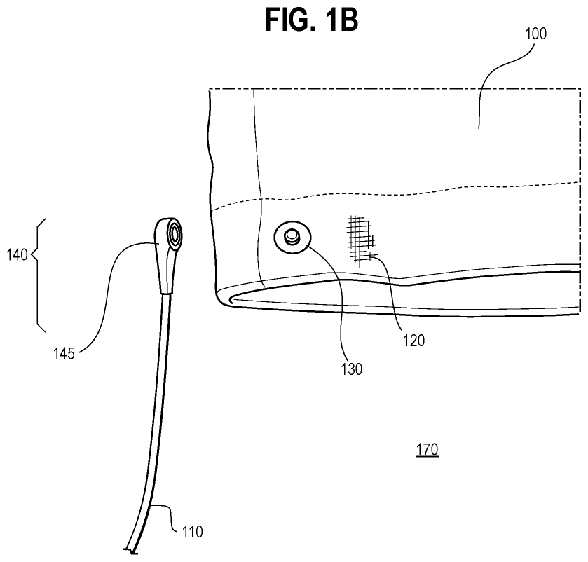

FIGS. 1A-1B are a set of two drawings of an electrically grounded hoodie 100.

FIG. 1A is a drawing of the electrically grounded hoodie 100. The electrically grounded hoodie 100 comprises a grounding wire 110 configured to provide electrical grounding. The electrically grounded hoodie 100 further comprises electrically conductive thread 120 configured to ground the entire garment through the grounding wire 110.

The electrically grounded hoodie 100 further comprises a conducting connector 130 configured to hold the grounding wire 110 tightly. For example, and as depicted, the conducting connector 130 comprises a button 130. The grounding wire 110 can be electrically connected to the conducting connector 130 at a first grounding wire end 140.

The grounding wire 110 comprises a grounding wire-clothing connector 145. As depicted, the grounding wire-clothing connector 145 comprises a snapping grounding wire-clothing connector 145 configured to snap tightly to the conducting connector 130. The grounding wire-clothing connector 145 is configured to electrically connect the grounding wire 110 to the conducting connector 130.

The grounding wire 110 can be electrically connected at a second grounding wire end 150 to an electrical ground 160.

The grounding wire 110 further comprises a grounding wire-ground connector 165 configured to hold the grounding wire 110 tightly. As depicted, the grounding wire-ground connector 165 holds the grounding wire 110 tightly by mating with the electrical ground 160. As depicted, the electrical ground 160 comprises a three-prong outlet 160.

Dotted line 170 in FIG. 1A is the boundary of a detail view of the electrically grounded hoodie 100 provided and discussed in more detail in FIG. 1B.

FIG. 1B shows a detail view of a portion 170 of FIG. 1A, showing a portion of the electrically grounded hoodie 100. Dotted line 170 in FIG. 1A is the boundary of the detail view of the device shown in FIG. 1B.

FIG. 1B depicts the conducting connector 130. The conducting connector 130 is configured to hold the grounding wire 110 tightly. As depicted, the conducting connector 130 mates with the grounding wire-clothing connector 145 at the first grounding wire end 140.

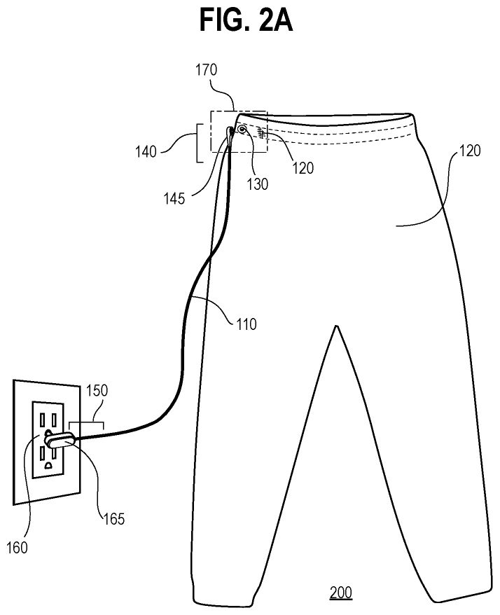

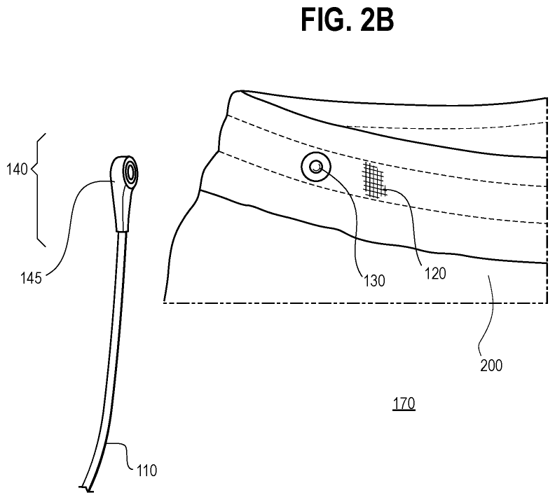

FIGS. 2A-2B are a set of two drawings of electrically grounded pants 200.

FIG. 2A is a drawing of the electrically grounded pants 200. The electrically grounded pants 200 comprises a grounding wire 110 configured to provide electrical grounding. The electrically grounded pants 200 further comprises electrically conductive thread 120 configured to ground the entire garment through the grounding wire 110.

The electrically grounded pants 200 further comprises a conducting connector 130 configured to hold the grounding wire 110 tightly. For example, and as depicted, the conducting connector 130 comprises a button 130. The grounding wire 110 can be electrically connected to the conducting connector 130 at a first grounding wire end 140.

The grounding wire 110 comprises a grounding wire-clothing connector 145. As depicted, the grounding wire-clothing connector 145 comprises a snapping grounding wire-clothing connector 145 configured to snap tightly to the conducting connector 130. The grounding wire-clothing connector 145 is configured to electrically connect the grounding wire 110 to the conducting connector 130.

The grounding wire 110 can be electrically connected at a second grounding wire end 150 to an electrical ground 160.

The grounding wire 110 further comprises a grounding wire-ground connector 165 configured to hold the grounding wire 110 tightly. As depicted, the grounding wire-ground connector 165 holds the grounding wire 110 tightly by mating with the electrical ground 160. As depicted, the electrical ground 160 comprises a three-prong outlet 160.

Dotted line 170 in FIG. 2A is the boundary of a detail view of the electrically grounded pants 200 provided and discussed in more detail in FIG. 2B.

FIG. 2B shows a detail view of a portion 170 of FIG. 2A, showing a portion of the electrically grounded pants 200. Dotted line 170 in FIG. 2A is the boundary of the detail view of the device shown in FIG. 2B.

FIG. 2B depicts the conducting connector 130. The conducting connector 130 is configured to hold the grounding wire 110 tightly. As depicted, the conducting connector 130 mates with the grounding wire-clothing connector 145 at the first grounding wire end 140.

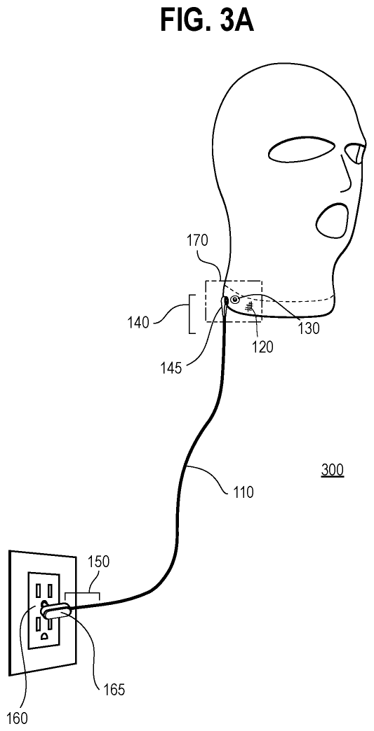

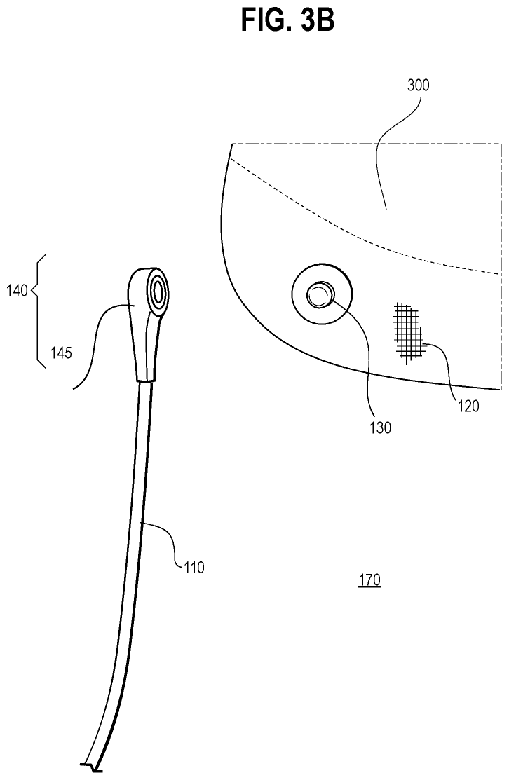

FIGS. 3A-3B are a set of two drawings of an electrically grounded ski mask 300.

FIG. 3A is a drawing of the electrically grounded ski mask 300. The electrically grounded ski mask 300 comprises a grounding wire 110 configured to provide electrical grounding. The electrically grounded ski mask 300 further comprises electrically conductive thread 120 configured to ground the entire garment through the grounding wire 110.

The electrically grounded ski mask 300 further comprises a conducting connector 130 configured to hold the grounding wire 110 tightly. For example, and as depicted, the conducting connector 130 comprises a button 130. The grounding wire 110 can be electrically connected to the conducting connector 130 at a first grounding wire end 140.

The grounding wire 110 comprises a grounding wire-clothing connector 145. As depicted, the grounding wire-clothing connector 145 comprises a snapping grounding wire-clothing connector 145 configured to snap tightly to the conducting connector 130. The grounding wire-clothing connector 145 is configured to electrically connect the grounding wire 110 to the conducting connector 130.

The grounding wire 110 can be electrically connected at a second grounding wire end 150 to an electrical ground 160.

The grounding wire 110 further comprises a grounding wire-ground connector 165 configured to hold the grounding wire 110 tightly. As depicted, the grounding wire-ground connector 165 holds the grounding wire 110 tightly by mating with the electrical ground 160. As depicted, the electrical ground 160 comprises a three-prong outlet 160.

Dotted line 170 in FIG. 3A is the boundary of a detail view of the electrically grounded ski mask 300 provided and discussed in more detail in FIG. 3B.

FIG. 3B shows a detail view of a portion 170 of FIG. 3A, showing a portion of the electrically grounded ski mask 300. Dotted line 170 in FIG. 3A is the boundary of the detail view of the device shown in FIG. 3B.

FIG. 3B depicts the conducting connector 130. The conducting connector 130 is configured to hold the grounding wire 110 tightly. As depicted, the conducting connector 130 mates with the grounding wire-clothing connector 145 at the first grounding wire end 140.

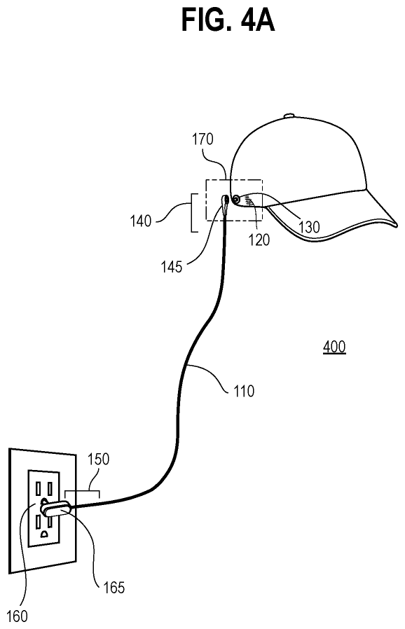

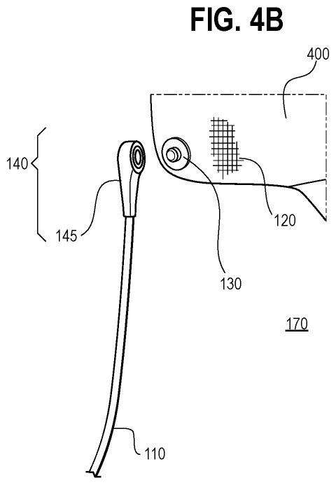

FIGS. 4A-4B are a set of two drawings of an electrically grounded cap 400.

FIG. 4A is a drawing of an electrically grounded cap 400. The electrically grounded cap 400 comprises a grounding wire 110 configured to provide electrical grounding. The electrically grounded cap 400 further comprises electrically conductive thread 120 configured to ground the entire garment through the grounding wire 110.

The electrically grounded cap 400 further comprises a conducting connector 130 configured to hold the grounding wire 110 tightly. For example, and as depicted, the conducting connector 130 comprises a button 130. The grounding wire 110 can be electrically connected to the conducting connector 130 at a first grounding wire end 140.

The grounding wire 110 comprises a grounding wire-clothing connector 145. As depicted, the grounding wire-clothing connector 145 comprises a snapping grounding wire-clothing connector 145 configured to snap tightly to the conducting connector 130. The grounding wire-clothing connector 145 is configured to electrically connect the grounding wire 110 to the conducting connector 130.

The grounding wire 110 can be electrically connected at a second grounding wire end 150 to an electrical ground 160.

The grounding wire 110 further comprises a grounding wire-ground connector 165 configured to hold the grounding wire 110 tightly. As depicted, the grounding wire-ground connector 165 holds the grounding wire 110 tightly by mating with the electrical ground 160. As depicted, the electrical ground 160 comprises a three-prong outlet 160.

Dotted line 170 in FIG. 4A is the boundary of a detail view of the electrically grounded cap 400 provided and discussed in more detail in FIG. 4B.

FIG. 4B shows a detail view of a portion 170 of FIG. 4A, showing a portion of the electrically grounded cap 400. Dotted line 170 in FIG. 4A is the boundary of the detail view of the device shown in FIG. 4B.

FIG. 4B depicts the conducting connector 130. The conducting connector 130 is configured to hold the grounding wire 110 tightly. As depicted, the conducting connector 130 mates with the grounding wire-clothing connector 145 at the first grounding wire end 140.

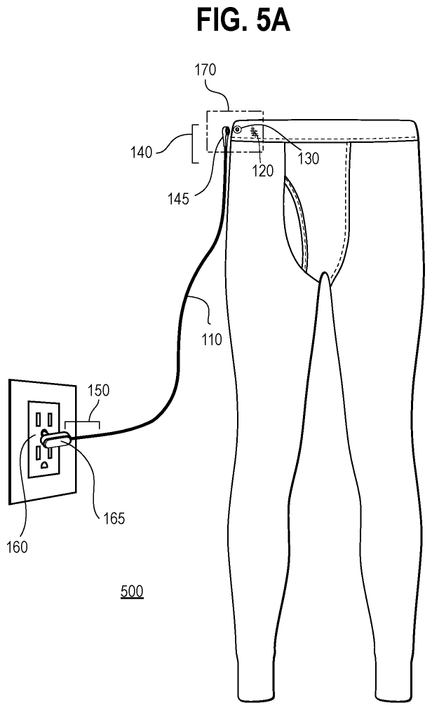

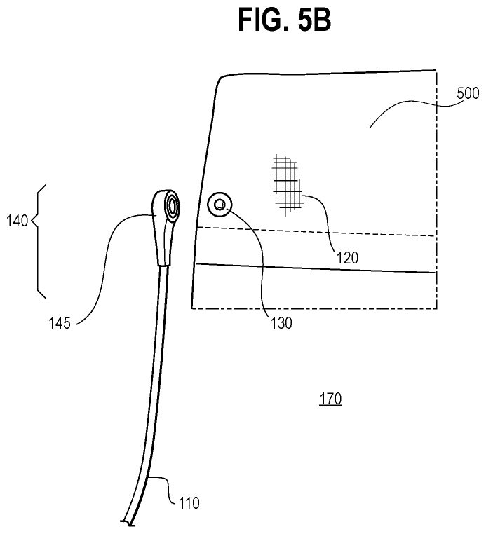

FIGS. 5A-5B are a set of two drawings of electrically grounded long underwear 100.

FIG. 5A is a drawing of an electrically grounded long underwear 500. The electrically grounded long underwear 500 comprises a grounding wire 110 configured to provide electrical grounding. The electrically grounded long underwear 500 further comprises electrically conductive thread 120 configured to ground the entire garment through the grounding wire 110.

The electrically grounded long underwear 500 further comprises a conducting connector 130 configured to hold the grounding wire 110 tightly. For example, and as depicted, the conducting connector 130 comprises a button 130. The grounding wire 110 can be electrically connected to the conducting connector 130 at a first grounding wire end 140.

The grounding wire 110 comprises a grounding wire-clothing connector 145. As depicted, the grounding wire-clothing connector 145 comprises a snapping grounding wire-clothing connector 145 configured to snap tightly to the conducting connector 130. The grounding wire-clothing connector 145 is configured to electrically connect the grounding wire 110 to the conducting connector 130.

The grounding wire 110 can be electrically connected at a second grounding wire end 150 to an electrical ground 160.

The grounding wire 110 further comprises a grounding wire-ground connector 165 configured to hold the grounding wire 110 tightly. As depicted, the grounding wire-ground connector 165 holds the grounding wire 110 tightly by mating with the electrical ground 160. As depicted, the electrical ground 160 comprises a three-prong outlet 160.

Dotted line 170 in FIG. 5A is the boundary of a detail view of the electrically grounded long underwear 500 provided and discussed in more detail in FIG. 5B.

FIG. 5B shows a detail view of a portion 170 of FIG. 5A, showing a portion of the electrically grounded long underwear 500. Dotted line 170 in FIG. 5A is the boundary of the detail view of the device shown in FIG. 5B.

FIG. 5B depicts the conducting connector 130. The conducting connector 130 is configured to hold the grounding wire 110 tightly. As depicted, the conducting connector 130 mates with the grounding wire-clothing connector 145 at the first grounding wire end 140.

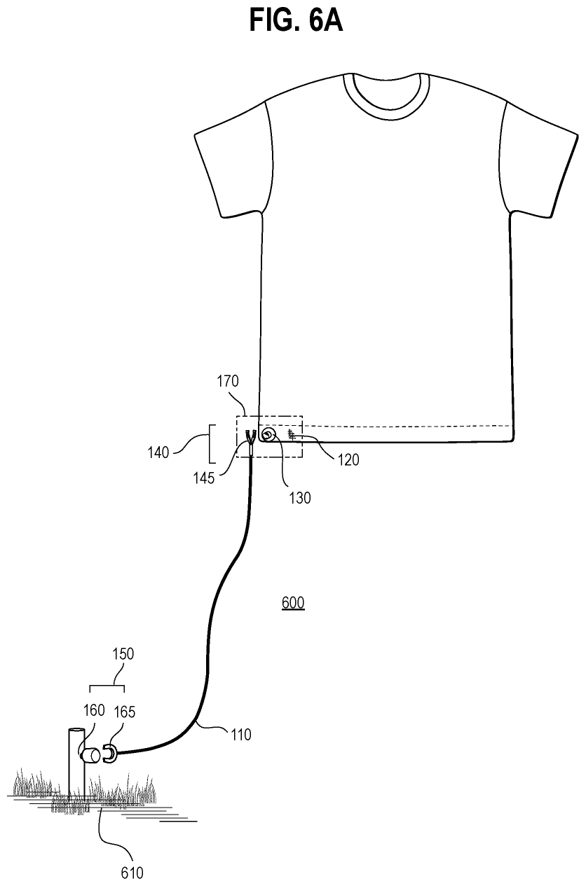

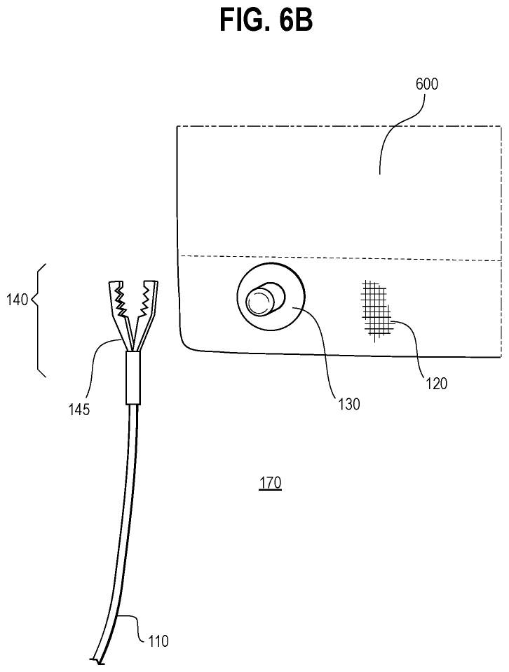

FIGS. 6A-6B are a set of two drawings of an electrically grounded shirt 600.

FIG. 6A is a drawing of an electrically grounded shirt 600. The electrically grounded shirt 600 comprises a grounding wire 110 configured to provide electrical grounding. The electrically grounded shirt 600 further comprises electrically conductive thread 120 configured to ground the entire garment through the grounding wire 110.

The electrically grounded shirt 600 further comprises a conducting connector 130 configured to hold the grounding wire 110 tightly to the electrically grounded shirt 600. For example, and as depicted, the conducting connector 130 comprises a clamp connector 130. The grounding wire 110 can be electrically connected to the conducting connector 130 at a first grounding wire end 140.

The grounding wire 110 optionally comprises a grounding wire-clothing connector 145. As depicted, the grounding wire-clothing connector 145 comprises a clamp 145 configured to snap tightly to the conducting connector 130. The grounding wire-clothing connector 145 is configured to electrically connect the grounding wire 110 to the conducting connector 130. Alternatively, or additionally, the conducting connector 130 attaches directly to the electrically grounded shirt 600.

The grounding wire 110 can be electrically connected at a second grounding wire end 150 to an electrical ground 160.

The grounding wire 110 further comprises a grounding wire-ground connector 165 configured to hold the grounding wire 110 tightly. As depicted, the grounding wire-ground connector 165 holds the grounding wire 110 tightly by mating with the electrical ground 160. As depicted, the electrical ground 160 comprises a direct ground 160 to earth 610.

Dotted line 170 in FIG. 6A is the boundary of a detail view of the electrically grounded shirt 600 provided and discussed in more detail in FIG. 1B.

FIG. 6B shows a detail view of a portion 170 of FIG. 6A, showing a portion of the electrically grounded shirt 600. Dotted line 170 in FIG. 6A is the boundary of the detail view of the device shown in FIG. 6B.

FIG. 6B depicts the conducting connector 130. The conducting connector 130 is configured to hold the grounding wire 110 tightly. As depicted, the conducting connector 130 mates with the grounding wire-clothing connector 145 at the first grounding wire end 140.

The electrically grounded blanket comprises a grounding wire configured to provide electrical grounding. The electrically grounded blanket further comprises electrically conductive thread configured to ground the entire blanket through the grounding wire. For example, the grounding wire has a length between approximately five feet and approximately twenty feet.

The electrically grounded blanket further comprises a conducting connector to which the grounding wire can be electrically connected at a first grounding wire end. The conducting connector is configured to hold the grounding wire tightly. Preferably, but not necessarily, the conducting connector is sewn into the electrically grounded blanket. Alternatively, the conducting connector is physically attached to the electrically grounded blanket.

For example, the conducting connector comprises a button sewn into the electrically grounded blanket. The button comprises a snap to which the grounding wire attaches. Alternatively, or additionally, the conducting connector comprises a clamp. Alternatively, or additionally, the conducting connector comprises another device other than a clamp or button configured to hold the grounding wire tightly.

The grounding wire is electrically connected at a second grounding wire end to an electrical ground.

For example, the electrical ground comprises one or more of a ground pin on a three-prong outlet, a ground pin on an outlet other than a three-prong outlet, a direct connection to the ground, and a connection to a grounding device comprised in an electrical appliance.

For example, the three-prong outlet comprises one or more of a ground fault circuit interrupter (GFCI) outlet, a ground fault interrupter (GFI) outlet, an arc fault circuit interrupter (AFCI) outlet, and a smart outlet.

The electrically grounded blanket further comprises electrically conductive thread configured to ground the entire blanket through the grounding wire. For example, the electrically conductive thread comprises one or more of silver, nickel, copper, gold, and another conductive material.

For example, the electrically conductive thread comprises high strength silver-coated nylon sewing thread manufactured by Suzhou Tek Silver Fiber Technology Co., Ltd. (http://mlsilver-fiber.com), of Suzhou, Jiangsu, China. For example, the electrically conductive thread can be used as a liner for electrically grounded pants.

Preferably, but not necessarily, the conducting connector is located near a center of the blanket.

For example, the conducting connector is preferably located within six inches of the center of the blanket. For example, the conducting connector is preferably located within twelve inches of the user's center of the blanket.

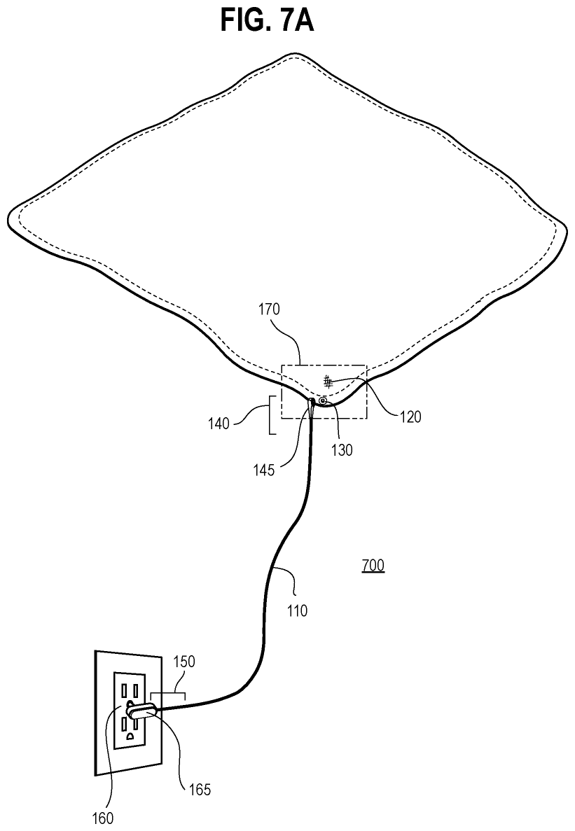

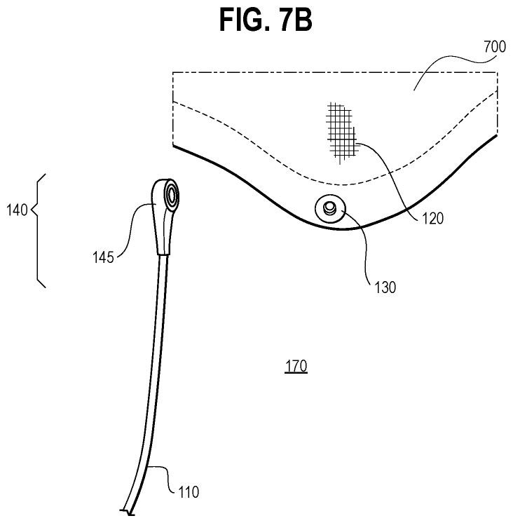

FIGS. 7A-7B are a set of two drawings of an electrically grounded blanket 700.

FIG. 7A is a drawing of the electrically grounded blanket 700.

The electrically grounded blanket 700 comprises a grounding wire 110 configured to provide electrical grounding. The electrically grounded blanket 700 further comprises electrically conductive thread 120 configured to ground the entire blanket through the grounding wire 110.

The electrically grounded blanket 700 further comprises a conducting connector 130 configured to hold the grounding wire 110 tightly. For example, and as depicted, the conducting connector 130 comprises a button 130. The grounding wire 110 can be electrically connected to the conducting connector 130 at a first grounding wire end 140.

The grounding wire 110 comprises a grounding wire-blanket connector 145. As depicted, the grounding wire-blanket connector 145 comprises a snapping grounding wire-blanket connector 145 configured to snap tightly to the conducting connector 130. The grounding wire-blanket connector 145 is configured to electrically connect the grounding wire 110 to the conducting connector 130.

The grounding wire 110 can be electrically connected at a second grounding wire end 150 to an electrical ground 160.

The grounding wire 110 further comprises a grounding wire-ground connector 165 configured to hold the grounding wire 110 tightly. As depicted, the grounding wire-ground connector 165 holds the grounding wire 110 tightly by mating with the electrical ground 160. As depicted, the electrical ground 160 comprises a three-prong outlet 160.

Dotted line 170 in FIG. 7A is the boundary of a detail view of the electrically grounded blanket 700 provided and discussed in more detail in FIG. 7B.

FIG. 7B shows a detail view of a portion 170 of FIG. 7A, showing a portion of the electrically grounded blanket 700. Dotted line 170 in FIG. 7A is the boundary of the detail view of the device shown in FIG. 7B.

FIG. 7B depicts the conducting connector 130. The conducting connector 130 is configured to hold the grounding wire 110 tightly. As depicted, the conducting connector 130 mates with the grounding wire-blanket connector 145 at the first grounding wire end 140.

An advantage of embodiments of the invention is that location of the grounding wire near a waist of the user provides a pivot point to which the grounding wire is connected that does not have a lot of movement.

A further advantage of embodiments of the invention is conveniently and affordably providing the user with all the protection afforded by electrical grounding.

While the above representative embodiments have been described with certain components in exemplary configurations, it will be understood by one of ordinary skill in the art that other representative embodiments can be implemented using different configurations and/or different components. For example, it will be understood by one of ordinary skill in the art that the order of certain steps and certain components can be altered without substantially impairing the functioning of the invention. It will be further understood by those of skill in the art that the number of variations of embodiments of the invention are virtually limitless. For example, other electrically grounded articles of clothing can be manufactured other than those specifically delineated in our examples. Virtually any imaginable electrically grounded clothing item can be manufactured while remaining within the scope of embodiments of the invention. For example, the electrically grounded clothing item comprises one or more of a onesie for a child, an adult onesie, and a thermal undershirt. For example, the thermal undershirt comprises a woolen thermal undershirt.

For example, the cord extending from the electrically grounded article of clothing to an electrical wall socket can have virtually any length. The suggested range of lengths is merely provided as a suggestion. For example, one or more of the electrically grounded article of clothing and the electrically grounded blanket can be electrically grounded by a method other than an electrically connection to an electrical wall socket. For example, the one or more of the electrically grounded article of clothing and the electrically grounded blanket can be electrically connected to a grounding device comprised in an electrical appliance. For example, the grounding device comprises one or more of a metal internal structure within the appliance, or external housing of the appliance.

The representative embodiments and disclosed subject matter, which have been described in detail herein, have been presented by way of example and illustration and not by way of limitation. It will be understood by those skilled in the art that various changes may be made in the form and details of the described embodiments resulting in equivalent embodiments that remain within the scope of the invention. It is intended, therefore, that the subject matter in the above description shall be interpreted as illustrative and shall not be interpreted in a limiting sense.

What is claimed is:

1. An electrically grounded clothing item, comprising:
a grounding wire configured to provide electrical grounding;
electrically conductive thread configured to ground the entire garment through the grounding wire; and
a conducting connector to which the grounding wire can be electrically connected at a first grounding wire end, the conducting connector configured to hold the grounding wire tightly to the electrically grounded clothing item, the grounding wire electrically connected at a second grounding wire end to an electrical ground.

2. The electrically grounded clothing item of claim 1, wherein the electrical ground comprises one or more of a ground pin on a three-prong outlet, a ground pin on an outlet other than a three-prong outlet, a direct connection to the ground, and a connection to a grounding device comprised in an electrical appliance.

3. The electrically grounded clothing item of claim 2, wherein the three-prong outlet comprises one or more of a ground fault circuit interrupter (GFCI) outlet, a ground fault interrupter (GFI) outlet, an arc fault circuit interrupter (AFCI) outlet, and a smart outlet.

4. The electrically grounded clothing item of claim 1, wherein the clothing item comprises one or more of a hoodie, pants, a shirt, a ski mask, a cap, and long underwear.

5. The electrically grounded clothing item of claim 1, wherein the conducting connector comprises one or more of a snap, a button and a clamp.

6. The electrically grounded clothing item of claim 1, wherein the conducting connector is sewn into the electrically grounded clothing.

7. The electrically grounded clothing item of claim 1, wherein the conducting connector is physically attached to the electrically grounded clothing.

8. The electrically grounded clothing item of claim 1, wherein the grounding wire has a length between approximately five feet and approximately twenty feet.

9. The electrically grounded clothing item of claim 1, wherein the connection between the grounding wire and the clothing item comprises one or more of a snap configured to connect to a button comprised in the clothing item and a clamp configured to grasp the grounding wire.

10. The electrically grounded clothing item of claim 6, wherein the conducting connector is located near a waist line of a user.

11. The electrically grounded clothing item of claim 1, wherein the clothing item comprises one or more of a shirt and a hoodie, and wherein the conducting connector is located near a bottom of the clothing item.

12. The electrically grounded clothing item of claim 10, wherein the conducting connector is located within six inches of a bottom of the electrically grounded clothing item.

13. The electrically grounded clothing item of claim 10, wherein the conducting connector is located within twelve inches of a bottom of the electrically grounded clothing item.

14. The electrically grounded clothing item of claim 1, wherein the clothing item comprises one or more of pants and long woolen underwear pants, and wherein the conducting connector is located near a top of the clothing item.

15. The electrically grounded clothing item of claim 13, wherein the conducting connector is located within six inches of a top of the electrically grounded clothing item.

16. The electrically grounded clothing item of claim 1, wherein the grounding wire comprises a grounding wire-clothing connector configured to electrically connect the grounding wire to the conducting connector.

17. The electrically grounded clothing item of claim 15, wherein the grounding wire-clothing connector comprises a snapping grounding wire-clothing connector configured to snap tightly to the conducting connector.

18. The electrically grounded clothing item of claim 15, wherein the grounding wire further comprises a grounding wire-ground connector configured to hold the grounding wire tightly by mating with the electrical ground.

19. An electrically grounded blanket, comprising:
a grounding wire configured to provide electrical grounding;
electrically conductive thread configured to ground the entire blanket through the grounding wire; and
a conducting connector to which the grounding wire can be electrically connected at a first grounding wire end, the conducting connector configured to hold the grounding wire tightly to the electrically grounded blanket, the grounding wire electrically connected at a second grounding wire end to an electrical ground.

20. The electrically grounded blanket of claim 19, wherein the conducting connector is located near a center of the blanket.
